(19) United States
(12) Patent Application Publication
(10) Pub. No.: US 2026/0356781 A1
(43) Pub. Date: Nov. 12, 2026

(54) PURIFICATION OF GLYCERIN AS EXCIPIENT IN PARENTERAL PHARMACEUTICAL APPLICATIONS

(71) Applicant: ARCHER-DANIELS-MIDLAND COMPANY, Decatur, IL (US)

(72) Inventors: Jose Leboreiro Hernandez, Decatur, IL (US); Lucas Kreidl, Decatur, IL (US); Lori Wicklund, Argenta, IL (US); Chi-Cheng Ma, Naperville, IL (US); Mitchell Schultz, Mt. Zion, IL (US)

(21) Appl. No.: 19/120,893

(22) PCT Filed: Oct. 10, 2023
(86) PCT No.: PCT/US2023/076488

§ 371 Date: Apr. 14, 2025

Related U.S. Application Data
(60) Provisional application No. 63/415,809, filed on Oct. 13, 2022.

(51) Int. Cl. C07C 29/90; A61K 47/10; C07C 29/80; C07C 31/22

(52) U.S. Cl. CPC C07C 29/90; A61K 47/10; C07C 29/80; C07C 31/225

(57) ABSTRACT

Methods are disclosed for the purification of a biobased glycerin feedstock, in particular but without limitation, a biobased glycerin feedstock already containing not more than 10 parts per million by weight in total of aldehydes, to provide a glycerin suitable for use in a parenteral pharmaceutical composition containing 5 parts per million or less of glyceraldehyde and 1 part per million or less of formaldehyde based on the weight of glycerin in the composition and further comprising at least one active pharmaceutical ingredient which would react with either or both of glyceraldehyde and formaldehyde if these were present at greater concentrations in the biobased glycerin component of the composition.

### CROSS-REFERENCE TO RELATED APPLICATIONS

This application is a national stage entry of International Application No. PCT/US2023/076488, filed Oct. 10, 2023, which itself claims priority to U.S. Provisional Patent Application No. 63/415,809, filed Oct. 13, 2022, the contents of each are incorporated herein by reference.

### FIELD OF THE INVENTION

The present invention relates to methods for the purification of glycerin.

### BACKGROUND OF THE INVENTION

It is well known that pharmaceutical ingredients have exacting purity requirements. Glycerin is a versatile material obtained from both plant and animal sources and from propylene from petroleum resources, and is used in medical, personal care as well as pharmaceutical applications.

U.S. Pat. No. 9,097,692, Milek et al., “Method for Quantitatively Determining Impurities in Glycerin”, provides an illustration of these exacting purity requirements, in relating that glycerin for use in certain pharmaceutical compositions such as, for example, a polypeptide described in EP 1242121 B1 (especially, a particular insulin), must according to the European Pharmacopeia include less than 10 parts per million of aldehyde impurities. Unfortunately, according to Milek et al., the method prescribed in the European Pharmocopeia for ascertaining compliance with this exacting purity standard is in fact incapable of reliably determining the true content of aldehydes and ketones in a supplier's glycerin, such that due to the reactivity of the same species, the quality of the finished pharmaceutical can be negatively affected and the finished pharmaceutical rendered noncompliant with the aldehydes standard. Accordingly, an alternative analytical method is described for ascertaining compliance, more particularly, providing a “means of which as many as possible and particular the entirety of impurities in the form of aldehydes and ketones can be better determined quantitatively”, col. 2, lines 6-8.

Remarkably, however, Milek et al. provide no description of a purification method by which glycerin determined by means of their analytical method to contain more than the prescribed maximum permissible content of aldehydes may then be made compliant with the European Pharmacopeia standard, nor describe how commercially-available glycerins obtained from different sources—for example, from hydrolysis of triglycerides on the one hand and from propylene from petroleum processing on the other—may differ in their impurities and consequently require different purification methods in order to be useful for this pharmaceutical application or that.

### SUMMARY

The following presents a simplified summary of the invention in order to provide a basic understanding of some of its aspects. This summary is not an extensive overview of the invention and is intended neither to identify key or critical elements of the invention nor to delineate its scope. The sole purpose of this summary is to present some concepts of the invention in a simplified form as a prelude to the more detailed description that is presented later.

With this understanding, the present invention in one aspect relates to the purification of glycerin, and in particular, biologically derived glycerin from the hydrolysis of triglycerides, for use as an excipient in a parenteral pharmaceutical composition.

In another, more particular aspect, the present invention relates to the purification of glycerin that contains 10 parts per million by weight or less of total aldehydes, so that the purified glycerin is characterized by a glyceraldehyde content of less than 5 parts per million by weight and a formaldehyde content of less than 1 part per million by weight.

In another aspect, the present invention relates to a parenteral pharmaceutical composition comprising a biobased glycerin component characterized by a glyceraldehyde content of less than 5 parts per million by weight (of the glycerin component) and a formaldehyde content of less than 1 part per million by weight, and further comprising at least one active pharmaceutical ingredient which would react with either or both of glyceraldehyde and formaldehyde if these were present at greater concentrations in the biobased glycerin component.

Parenthetically and for ease of understanding, as used in this application the singular forms “a”, “an” and “the” include plural references unless the context clearly indicates otherwise. The term “comprising” and its derivatives, as used above and elsewhere herein, are similarly intended to be open ended terms that specify the presence of the stated features, elements, components, groups, integers, and/or steps, but do not exclude the presence of other unstated features, elements, components, groups, integers and/or steps. This understanding also applies to words having similar meanings, such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers, and/or steps. The term “consisting essentially of”, as used herein, is intended to specify the presence of the stated features, elements, components, groups, integers, and/or steps, as well as those that do not materially affect the basic and novel characteristic(s) of stated features, elements, components, groups, integers, and/or steps.

“Biobased” as used herein, it should be noted, means and refers to those materials whose carbon content is shown by ASTM D6866 to be derived from or based in significant part (at least 20 percent or more) upon biological products or renewable agricultural materials (including but not being limited to plant, animal and marine materials) or forestry materials.

In this respect ASTM Method D6866, similar to radiocarbon dating, compares how much of a decaying carbon isotope remains in a sample to how much would be in the same sample if it were made of entirely recently grown materials. The percentage is called the biobased content of the product. Samples are combusted in a quartz sample tube and the gaseous combustion products are transferred to a borosilicate break seal tube. In one method, liquid scintillation is used to count the relative amounts of carbon isotopes in the carbon dioxide in the gaseous combustion products. In a second method, 13C/12C and 14C/12C isotope ratios are counted (14C) and measured (13C/12C) using accelerator mass spectrometry. Zero percent 14C indicates the entire lack of 14C atoms in a material, thus indicating a fossil (for example, petroleum based) carbon source. One hundred percent 14C, after correction for the post-1950 bomb injection of 14C into the atmosphere, indicates a modern carbon source. ASTM D6866 effectively distinguishes between biobased materials and petroleum derived materials in part because isotopic fractionation due to physiological processes, such as, for example, carbon dioxide transport within plants during photosynthesis, leads to specific isotopic ratios in natural or biobased compounds. By contrast, the 13C/12C carbon isotopic ratio of petroleum and petroleum derived products is different from the isotopic ratios in natural or bioderived compounds due to different chemical processes and isotopic fractionation during the generation of petroleum. In addition, radioactive decay of the unstable 14C carbon radioisotope leads to different isotope ratios in biobased products compared to petroleum products.

A particular biobased glycerin feedstock of interest is that which is presently obtained commercially by the hydrolysis of triglycerides to separate fatty acids from a glycerin backbone.

The aforementioned and other aspects, embodiments, and associated advantages will become apparent from the following Detailed Description.

### DETAILED DESCRIPTION OF EMBODIMENTS

In a first aspect, the present invention concerns the purification of glycerin, and in particular, a biobased glycerin from the processing of oilseeds, to provide a material with the requisite purity to be included in a parenteral pharmaceutical composition alongside one or more active pharmaceutical ingredients.

In certain embodiments, a biobased, commercially available glycerin, including but not being limited to a biobased, commercially available glycerin which already meets the purity requirement for total aldehydes under the European Pharmacopoeia-namely, a biobased glycerin which has been determined as prescribed by the European Pharmacopoeia as containing not more than 10 parts per million of aldehydes (under the EP monograph in place as of the filing of the instant application), is purified by means described and exemplified hereafter so that the glyceraldehyde content of the pharma-grade biobased glycerin is reduced to less than 5 parts per million by weight and the formaldehyde content of the glycerin is reduced to less than 1 part per million by weight.

In other more particular embodiments, a biobased glycerin determined according to the different analytical method described by Milek et al. or by another analytical method or combination of methods as containing not more than a maximum prescribed content of total aldehydes per any relevant governing monograph is purified so that the glyceraldehyde content following the purification method is less than 5 parts per million by weight and the formaldehyde content is less than 1 part per million by weight. In this regard, as of the filing of the instant application only the European Pharmacopoeia contains a specification for aldehydes, at 10 parts per million or less by weight of total aldehydes.

In one embodiment, chemical base treatment is used to reduce the amounts of glyceraldehyde and formaldehyde present in a biobased glycerin, even including (but not being limited thereto) a biobased glycerin already containing 10 parts per million or less by weight of total aldehydes, to below the prescribed limits of 5 parts per million by weight and 1 part per million by weight. Examples of suitable bases include sodium hydroxide, potassium hydroxide and sodium borohydride, with sodium hydroxide being preferred. An exemplary process would involve combining 1 percent by weight of sodium hydroxide with the glycerin to be treated and with agitation, for example, in a stirred tank reactor, for from one to eight hours at 60 degrees Celsius and under reduced oxygen conditions (for example, by applied vacuum or with a nitrogen purge, to inhibit uptake of water from ambient air and oxidation of aldehydes).

In another embodiment, molecular or short path distillation, for example, using wiped film evaporation, is used. In one simple apparatus and method, a wiped film evaporator is used to process glycerin to be further purified at 120 degrees Celsius and under 3 torrs of vacuum, with a recycle loop as needed to further reduce the aldehydes. In another apparatus and associated method, glycerin to be purified is fed into a packed column operating at 120 degrees Celsius and under 3 torrs of vacuum, with the aldehydes and some glycerin being taken overhead while the bottoms glycerin from the column containing reduced aldehydes is processed in a finishing, wiped film evaporator to provide the desired purified glycerin product.

In another embodiment, ion exchange chromatography is used. Preferred resins are generally weak base anion exchange resins with styrenic, phenolic and macroporous structures. Especially preferred commercially available examples include the resins sold under Thermax Tulsion A-2X MP macroporous weak base anion exchange resin with a polystyrene copolymer matrix structure and tertiary amine functional group (Thermax Inc, Houston TX), Lewatit VP OC 1065 macroporous, divinyl benzene crosslinked polymer in spherical bead form with primary amine groups (Lanxess AG, Cologne, Germany) and Purolite A133S macroporous polystyrene crosslinked with divinylbenzene with tertiary amine functional groups, weak base anion resin (Purolite Corporation, King of Prussia, PA). We have also had success with nonfunctionalized resins, for example, a nonionic, macroporous crosslinked divinylbenzene polymer such as sold by DuPont de Nemours, Inc. as Amberlite XAD-16N polymeric adsorbent resin.

In another embodiment, the purification is accomplished by contacting the glycerin with activated carbon. Preferred carbons are those specifically exemplified below. The manner of contacting can involve preferably simply combining powdered or granular activated carbon with a glycerin feedstock to be purified with continuous mixing for a time and preferably at an elevated temperature under reduced oxygen conditions, for example, about 24 hours, then cooling and filtering the glycerin/treating carbon mixture to recover the desired reduced aldehyde glycerin product. Alternatively, the glycerin feedstock could be continuously processed through one or more carbon beds or columns in series, containing/using one or more treatment carbons.

In another embodiment, steam stripping or deodorization is used to perform the purification. Application of from 1.8 percent to 5.8 percent by weight of steam, based on the weight of glycerin feedstock treated thereby, under vacuum for a period of time—for example, from two to four hours' contact time—would be an example of a suitable deodorization process.

In another embodiment, a biobased glycerin is subjected to treatment with hydrogen in the presence of a catalyst to provide a purified glycerin having at least a reduced content of at least one aldehyde compared to the starting biobased glycerin. In particular, a purified biobased glycerin having a reduced content of one or both of glyceraldehyde and formaldehyde is contemplated.

In still other embodiments, a combination of two or more of these methods is employed, for example, a combination of deodorization followed by carbon treatment.

The following examples are set forth as representative of the present invention. These examples are illustrative and not to be construed as limiting the scope of the invention as defined in the appended claims.

### Examples 1-5

10 g samples of a glycerin containing 18.4 parts per million by weight of glyceraldehyde were placed in vials with different amounts of a 1 weight percent sodium hydroxide solution, then the vials were capped and left to react for designated times at 60 degrees Celsius, with the glyceraldehyde content after such times being measured in the same way as the starting concentration was originally determined and with the results reported below in Table 1:

TABLE 1

| Amount Added | Rxn Time | GA, ppm |
|---|---|---|
| 20 microliters | 1 hr | 16.4 |
| 20 microliters | 4.5 hrs | 12.7 |
| 20 microliters | 8 hrs | 12.2 |
| 12.5 microliters | 1 hr | 18.2 |
| 12.5 microliters | 4.5 hrs | 15.2 |

### Examples 6-10

A 1-liter glass round-bottom flask with a heating mantle was provided with a connected elevated flask containing water for steam generation and with a connected vacuum pump to pull a vacuum on the 1-liter glass flask, and further with a condenser filled with dry ice to collect removed aldehydes. Two hundred grams of glycerin initially containing 13.7 ppm by weight of glyceraldehyde and 1.04 ppm by weight of formaldehyde were placed in the 1-liter glass flask for each deodorization experiment, with heating to 120 degrees Celsius or 130 degrees Celsius as indicated below in Table 2 and with 3 torr absolute of applied vacuum and with sparging steam through the flask. After the specified times, the application of heating, vacuum and steam was discontinued, and the flask's contents allowed to return to ambient (room) temperature and pressure. The remaining glyceraldehyde and formaldehyde in the deodorized glycerin were then quantitated for comparison to their initial concentrations, with the results shown below in Table 2 where “GA” is glyceraldehyde and “FA” is formaldehyde:

TABLE 2

| Glycerin | 200 g | 200 g | 200 g | 200 g | 200 g |
|---|---|---|---|---|---|
| T °C | 120 | 130 | 130 | 130 | 130 |
| Vac Torr | 3 | 3 | 3 | 3 | 3 |
| Steam | 3 | 2.66 | 2.8 | 2.5 | 3 |
| Rate, % | | | | | |
| Time (min) | 30 | 30 | 60 | 90 | 120 |
| GA, ppmw | 12.3 | 10.2 | 6.7 | 5.41 | 3.76 |
| FA, ppmw | 0.754 | 0.636 | 0.325 | 0.758 | 0.668 |

### Examples 11-24

Quantities of two different activated carbons were dried in an oven overnight at 110 to 120 degrees Celsius and then cooled to ambient temperature. The first carbon investigated was a low ash (<5 wt percent), 900 minimum mg/g iodine value, coconut-based carbon—OLC 12×30 from Calgon Carbon Corporation, Pittsburgh, PA (“OLC” in the Table following)—while the second carbon, PICACTIF Medicinal EP 40 from Jacobi Carbons AB, Kalmar, Sweden (“EP 40” in the Table following), was also a low ash, coconut based, steam-activated carbon characterized as having a particle size between 8 and 35 micrometers and further characterized by its manufacturer as being compliant with both the US Pharmacopeia and the European Pharmacopoeia. Varying quantities of each dried carbon were combined with 30 grams of an untreated glycerin characterized by a beginning glyceraldehyde and formaldehyde content in a series of 50 mL centrifuge tubes, which tubes were sealed and incubated at 40 degrees Celsius with mixing in a rotating rack for 24 hours. The carbon was then recovered from each such centrifuge tube by filtering, and the recovered treated glycerin then analyzed for its remaining glyceraldehyde and formaldehyde contents. Results are shown in Table 3 below:

TABLE 3

| Carbon Type | Loading, wt % | GA, ppmw | FA, ppmw |
|---|---|---|---|
| OLC | 0 | 24.4 | 0.844 |
| OLC | 0.024 | 24.9 | 0.96 |
| OLC | 0.014 | 24.9 | 1.11 |
| OLC | 0.107 | 21.7 | 1.37 |
| OLC | 0.214 | 12.3 | 1.22 |
| OLC | 0.403 | 6.28 | 0.925 |
| OLC | 0.799 | 3.36 | 0.904 |
| EP 40 | 0 | 23.8 | 0.336 |
| EP 40 | 0.059 | 21.4 | <LOQ |
| EP 40 | 0.107 | 20.9 | <LOQ |
| EP 40 | 0.204 | 19.3 | <LOQ |
| EP 40 | 0.421 | 16.6 | <LOQ |
| EP 40 | 0.803 | 14.0 | <LOQ |
| EP 40 | 1.605 | 10.4 | <LOQ |
| EP 40 | 3.177 | 10.0 | <LOQ |
| EP 40 | 6.391 | 11.5 | <LOQ |

### Examples 25 and 26

Stainless steel, 30 cubic centimeter volume tubular reactors with an internal diameter of 0.61 inches were used to evaluate catalytic hydrogenation as a means for reducing the content of at least certain aldehydes in a commercially available biobased glycerin. The reactors were jacketed and heated with circulating oil. Reactor temperatures were monitored via an internal thermocouple. Inlets of the reactors were attached to an Isco dual piston pump and mass flow controllers for supplying gases. Reactor outlets were attached to condensers kept at 5 degrees Celsius by a chiller unit.

Catalysts comprising 2% by weight loading ruthenium on an activated carbon powder and 1% by weight loading palladium on an activated carbon powder were evaluated for their effectiveness using a hydrogen flow rate of 0.4 mL/minute and pressure of 1800 psig (unless otherwise indicated in Table 4 below) and at an LHSV of 1 at reducing the observed content of various impurities in the glycerin, under the reactor temperatures and reaction times set forth below:

TABLE 4

| | 2- | 1- | Diethylene | | |
|---|---|---|---|---|---|
| | Monochloroacetin | Monochloroacetin | Glycol | Glyceraldehyde | Formaldehyde |
| Catalyst, Conditions | (mg/kg) | (mg/kg) | (mg/kg) | (μg/g) | (μg/g) |
| None* | 61.6 | 411 | 4.5 | 39.3 | 1.32 |
| Ru/C, 6 hrs | 47.7 | 305 | 2.8 | 1.79 | 0.195 |
| Ru/C, 78 hrs. | 55.8 | 379 | 3.5 | 11.5 | 0.345 |
| Ru/C, 400 psig, 24 | 57.6 | 386 | — | — | — |
| hrs. | | | | | |
| Pd/C, 120 deg. C, | 53.2 | 381 | — | — | — |
| 1200 psig, 12 hrs | | | | | |

*Starting glycerin feed

### Examples 27 Through 34

A 1-liter glass round-bottom flask with a heating mantle was provided with a connected elevated flask containing water for steam generation and with a connected vacuum pump to pull a vacuum on the 1-liter glass flask, and further with a condenser filled with dry ice to collect removed impurities from various lots of biobased glycerin containing different concentrations of both aldehydes and ketones (which interact in a similar fashion as aldehydes, in respect of a parenteral grade glycerin product).

For these examples, from nine hundred to one thousand grams of biobased glycerin initially containing up to 29.0 ppm by weight of glyceraldehyde, up to 0.73 ppm by weight of formaldehyde, up to 4.88 ppm by weight of hydroxyacetone, and up to 21.8 ppm by weight of dihydroxyacetone were placed in the 1-liter glass flask for each deodorization experiment, with heating to 130 degrees as Celsius indicated below in Table 5 and with 3 torr absolute of applied vacuum and with sparging steam through the flask. After the specified times, the application of heating, vacuum and steam was discontinued, and the flask's contents allowed to return to ambient (room) temperature and pressure. The remaining glyceraldehyde, formaldehyde, hydroxyacetone, and dihydroxyacetone in the deodorized glycerin were then quantitated for comparison to their initial concentrations, with the results shown below in Table 5 where “GA” is glyceraldehyde, “FA” is formaldehyde, “HA” is hydroxyacetone, and “DHA” is dihydroxyacetone:

TABLE 5

| Glycerin | | 1163 g | 1081 g | 1163 g | 1178 g | 1108 g | 1078 g | 1085 g | 1108 g |
|---|---|---|---|---|---|---|---|---|---|
| Steam Rate, % | | 7.6 | 5.1 | 5.8 | 4.5 | 4.4 | 5.5 | 8.6 | 8.4 |
| Time (min) | | 180 | 180 | 180 | 180 | 180 | 240 | 420 | 420 |
| GA, ppmw | | Starting | 29.01 | 7.41 | 10.08 | 24.16 | 8.07 | 6.53 | 17.56 |
| | | Glycerin | | | | | | | |
| Time (min) | | Deodorized | 7.41 | 2.90 | 1.34 | 8.07 | 3.29 | 1.71 | 3.51 |
| | | Glycerin | | | | | | | |
| FA, | Starting | 0.73 | 0.40 | 0.71 | 0.61 | 0.34 | 0.23 | 0.58 | 20.85 |
| ppmw | Glycerin | | | | | | | | |
| | Deodorized | 0.40 | 0.20 | 0.32 | 0.34 | 0.21 | 0.20 | 0.37 | 1.57 |
| | Glycerin | | | | | | | | |
| HA, | Starting | 3.89 | <0.5 | <0.5 | 2.47 | <0.5 | 0.36 | 3.53 | 0.67 |
| ppmw | Glycerin | | | | | | | | |
| | Deodorized | <0.5 | <0.5 | <0.5 | <0.5 | <0.5 | 0.58 | 0.20 | 0.23 |
| | Glycerin | | | | | | | | |
| DHA, | Starting | 21.8 | 5.9 | 4.7 | 17.5 | 7.7 | 3.90 | 11.23 | 4.88 |
| ppmw | Glycerin | | | | | | | | |
| | Deodorized | 5.9 | 2.1 | 1.1 | 7.7 | 3.2 | 1.02 | 0.88 | 0.33 |
| | Glycerin | | | | | | | | |

What is claimed is:

1. A process for providing a biobased glycerin suitable for use in a parenteral pharmaceutical composition containing one or more active pharmaceutical ingredients, comprising:
providing a biobased glycerin feedstock; and
applying to the glycerin feedstock one or more purification methods selected from the group consisting of chemical base treatment, catalytic hydrogenation, molecular distillation, ion exchange chromatography, deodorization and treatment with activated carbon, to produce a purified glycerin product containing 5 parts per million or less of glyceraldehyde and 1 part per million or less of formaldehyde.

2. The process of claim 1, wherein the biobased glycerin feedstock is characterized by a total aldehydes content of not more than 10 parts per million by weight.

3. The process of claim 1 or claim 2, wherein a combination of deodorization and treatment with activated carbon is employed.

4. A parenteral pharmaceutical composition, comprising a biobased glycerin component characterized by a glyceraldehyde content of less than 5 parts per million by weight of the glycerin component and a formaldehyde content of less than 1 part per million by weight on the same basis, and further comprising at least one active pharmaceutical ingredient which would react with either or both of glyceraldehyde and formaldehyde if these were present at greater concentrations in the biobased glycerin component.
